(19) United States
(12) Patent Application Publication
(10) Pub. No.: US 2007/0244348 A1
(43) Pub. Date: Oct. 18, 2007

(54) Process for producing olefin product from syngas

(76) Inventors: Michel Molinier, Houston, TX (US); James R. Lattner, LaPorte, TX (US); Matthew James Vincent, Baytown, TX (US); Kun Wang, Bridgewater, NJ (US)

Correspondence Address: ExxonMobil Chemical Company;Law Technology, P.O. Box 2149, Baytown, TX, 77522-2149, US

(21) Appl. No.: 11/711,321

(22) Filed: Feb. 26, 2007

Related U.S. Application Data
(60) Provisional application No. 60/791,770, filed on Apr. 13, 2006.

(51) Int. Cl. C07C 6/00

(52) U.S. Cl. 585/310

(57) ABSTRACT

This invention is directed to a process for making an olefin product from a mixed alcohol feed stream. The alcohol product that is formed using this invention contains significant quantities of methanol and ethanol, and is relatively in higher alcohols (i.e., C₃+ alcohols) and in branched alcohols. One of the catalysts used to form the mixed alcohol is an oxide-containing catalyst that has been modified to contain a Fischer-Tropsch metal (i.e., cobalt, iron, and nickel).

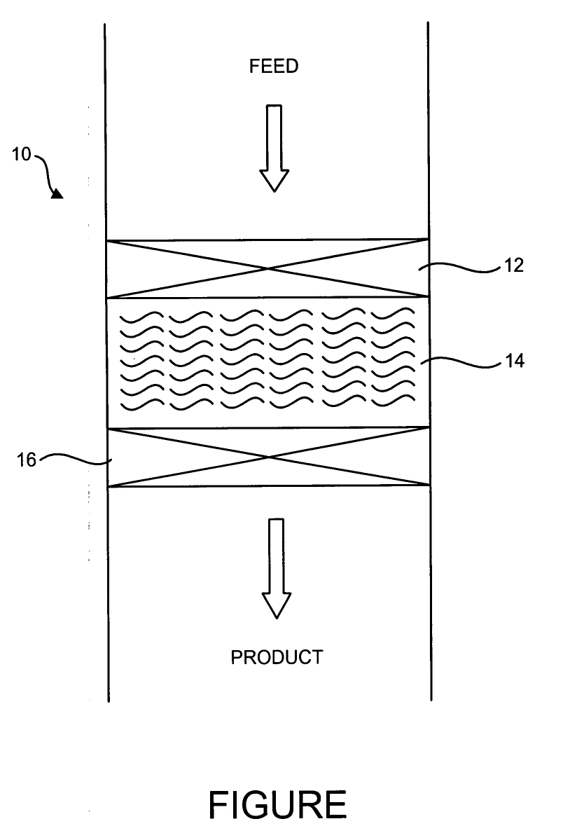

### CROSS REFERENCE TO RELATED APPLICATIONS

This claims the benefit of and priority from U.S. Ser. No. 60/791,770, filed Apr. 13, 2006. The above application is fully incorporated herein by reference.

### FIELD OF THE INVENTION

This invention is directed to a method of producing a mixed alcohol product from syngas, use of the alcohol product as a feedstock, and catalyst for use in the production of the alcohol product. The alcohol product is particularly useful in the manufacture of ethylene and propylene.

### BACKGROUND OF THE INVENTION

Ethylene and propylene are chemical compounds that are used quite extensively as feed components in the manufacture of a fairly wide array of more complex chemical compositions. For example, ethylene is predominantly used as a feed compound in the production of low and high density polyethylene products. Approximately 60% of world ethylene consumption goes into making polyethylene for such products as plastic films, containers, and coatings. Other uses include the production of vinyl chloride, ethylene oxide, ethylbenzene, and alcohols. Presently, about 90% of the ethylene is produced by steam cracking petroleum-based feedstocks such as light paraffin, naphtha, and gas oil.

About 55% of the world consumption of propylene is directed to the production of polypropylene. Other important end products include acrylonitrile for acrylic and nylon fibers, and propylene oxide for polyurethane foams. About two-thirds of the propylene is produced from steam cracking petroleum feedstock, and the remaining third as a by-product of FCC gasoline refining.

A potential alternative to producing ethylene and propylene from petroleum-based feedstocks is to use methanol. Methanol is typically produced from synthesis gas, and synthesis gas is typically derived from the reforming of natural gas.

U.S. Pat. No. 4,499,327 (Kaiser) discloses making olefins from methanol using a variety of SAPO molecular sieve catalysts. The advantage of using SAPO-based catalysts, particularly SAPO-34 based catalysts, is that such catalysts produce a substantially large amount of ethylene and propylene relative to other oxygenated hydrocarbons, e.g., alcohols, ethers, etc.

U.S. Pat. No. 6,518,475 (Xu) discloses a process of increasing ethylene selectivity in the conversion of a methanol-based feed. The method includes contacting a silicoaluminophosphate molecular sieve catalyst with a methanol composition that contains from about 1% to about 15% by weight acetone, and separating the ethylene and propylene from the olefin product. The use of the particular feed composition increases the amount of ethylene produced relative to that when pure methanol is used as the feed.

U.S. Patent Publication No. 2004/0116757 (Van Egmond) discloses a methanol-based composition, a method of making the composition, and a method of using the composition as a feedstock. The methanol composition contains methanol as a primary compound, but also includes one or more other alcohols such as ethanol and/or one or more aldehyde compounds. The methanol composition serves as a particularly desirable feed stream for use in the manufacture of olefins such as ethylene and propylene. Such feed streams result in increased production of ethylene or in the increased production of both ethylene and propylene.

U.S. Patent Publication No. 2005/0107482 (Van Egmond) discloses a process for producing light olefins from methanol and ethanol in a mixed alcohol stream. In one embodiment, the invention includes directing a first syngas stream to a methanol synthesis zone to form methanol and directing a second syngas stream and methanol to a homologation zone to form ethanol. The methanol and ethanol are sent to an oxygenate-to-olefin reaction system for conversion to ethylene and propylene.

Although mixed alcohol streams are desirable for use as feed streams in the production of olefins such as ethylene and propylene, simpler processes are needed. In particular, processes that reduce the complexity of coupling together mixed alcohol production with olefin formation are particularly desirable.

### SUMMARY OF THE INVENTION

This invention provides a process for producing a mixed alcohol stream and using that stream as a feedstock for the manufacture of olefins. In particular, the reactor system for making the mixed methanol feed is particularly uncomplicated in design, and can be easily coupled to a reaction system capable of forming olefins.

According to one aspect of the invention, there is provided a process for producing olefin product from syngas. The process includes a step of contacting at least two different alcohol forming catalysts with a syngas feed to form an alcohol product, such that at least one of the catalysts is an oxide-containing catalyst that has been modified with a Fischer-Tropsch metal. The alcohol product is then contacted with an olefin forming catalyst to form the olefin product.

In one embodiment, the oxide-containing catalyst that has been modified with a Fischer-Tropsch metal includes an oxide of at least one element selected from the group consisting of copper, silver, zinc, boron, magnesium, aluminum, vanadium, chromium, manganese, gallium, palladium, osmium, and zirconium. Preferably, the oxide-containing catalyst that has been modified with a Fischer-Tropsch metal is a Fischer-Tropsch metal modified copper-based oxide catalyst that includes an oxide of at least one element selected from the group consisting of silver, zinc, boron, magnesium, aluminum, vanadium, chromium, manganese, gallium, palladium, osmium, and zirconium. More preferably, the oxide-containing catalyst that has been modified with a Fischer-Tropsch metal is a Fischer-Tropsch metal modified copper-based oxide catalyst that includes an oxide of at least one element selected from the group consisting of zinc, magnesium, aluminum, chromium, and zirconium. Still more preferably, the oxide-containing catalyst that has been modified with a Fischer-Tropsch metal is a Fischer-Tropsch metal modified copper-based oxide catalyst that includes zinc oxide.

In another aspect of the invention, the Fischer-Tropsch metal is cobalt.

In yet another aspect, the oxide-containing catalyst that has been modified with a Fischer-Tropsch metal is downstream of at least one other alcohol forming catalyst. Preferably, another of the alcohol forming catalysts includes an oxide of at least one element selected from the group consisting of copper, silver, zinc, boron, magnesium, aluminum, vanadium, chromium, manganese, gallium, palladium, osmium, and zirconium. More preferably, another of the alcohol forming catalysts is a copper-based catalyst that includes an oxide of at least one element selected from the group consisting of silver, zinc, boron, magnesium, aluminum, vanadium, chromium, manganese, gallium, palladium, osmium, and zirconium. Still more preferably, another of the alcohol forming catalysts is a copper-based catalyst that includes an oxide of at least one element selected from the group consisting of zinc, magnesium, aluminum, chromium, and zirconium.

### BRIEF DESCRIPTION OF THE DRAWING

The attached FIGURE represents merely one aspect of the invention. The FIGURE is intended to be viewed as merely one of numerous embodiments within the scope of the overall invention as claimed. Specifically, the FIGURE is a flow diagram using two catalysts to form the alcohol product of the invention.

### DETAILED DESCRIPTION OF THE INVENTION

### I. Manufacture of Mixed Alcohol and Olefin Product

This invention is directed to a process for making an olefin product from a mixed alcohol feed stream. The mixed alcohol feed stream is made from a synthesis gas (syngas) feed stream. The syngas feed is converted to the mixed alcohol stream using at least two different catalysts, preferably using a dual catalyst system.

The alcohol product that is formed using this invention contains significant quantities of methanol and ethanol. The product is low in higher alcohols (i.e., C₃+ alcohols) and in branched alcohols.

One of the catalysts used in this invention is an oxide-containing catalyst that contains or has been modified to contain a Fischer-Tropsch metal (i.e., cobalt, iron, and nickel). Such a catalyst does not require the use of any promoter or ligand. This reduces the risk of alcohol and olefin product contamination.

In one embodiment of the invention, first and second alcohol forming catalysts are contacted with a syngas feed to form an alcohol product, and the alcohol product is contacted with an olefin forming catalyst to form the olefin product. Preferably, the second catalyst is the oxide-containing catalyst that has been modified to contain a Fischer-Tropsch metal.

### II. Synthesis Gas Production

### A. Methods of Making Synthesis Gas Feed

According to this invention, synthesis gas (syngas) is used as feed to make a mixed alcohol stream, and the mixed alcohol stream is then converted to olefin. Synthesis gas comprises carbon monoxide and hydrogen. Optionally, carbon dioxide and nitrogen are included.

Synthesis gas can be manufactured from a variety of carbon sources. Examples of such sources include biomass, natural gas, C₁-C₅ hydrocarbons, naphtha, heavy petroleum oils, or coke (i.e., coal). Preferably, the hydrocarbon feed stream comprises methane in an amount of at least about 50% by volume, more preferably at least about 70% by volume, most preferably at least about 80% by volume. In one embodiment of this invention natural gas is the preferred hydrocarbon feed source.

Any suitable syngas forming reactor or reaction system can be used in combination with the fluidized-bed reaction system of this invention. Examples of synthesis gas forming systems include partial oxidation, steam or CO₂ reforming, or some combination of these two chemistries.

### B. Steam Reforming to Make Syngas

In the catalytic steam reforming process, hydrocarbon feeds are converted to a mixture of H₂, CO, and CO₂ by reacting hydrocarbons with steam over a catalyst. This process involves the following reactions:

CH₄+H₂O [symbol] CO+3H₂ (1)

or

CₙHₘ+nH₂O [symbol] nCO+[n+(m/2)]H₂ (2)

and

CO+H₂ [symbol] CO₂+H₂ (3) (shift reaction)

The reaction is carried out in the presence of a catalyst. Any conventional reforming type catalyst can be used. The catalyst used in the step of catalytic steam reforming comprises at least one active metal or metal oxide of Group 6 or Group 8-10 of the Periodic Table of the Elements. The Periodic Table of the Elements referred to herein is that from CRC Handbook of Chemistry and Physics, 82^{nd} Edition, 2001-2002, CRC Press LLC, which is incorporated herein by reference.

In one embodiment, the catalyst contains at least one Group 6 or Group 8-10 metal, or oxide thereof, having an atomic number of 28 or greater. Specific examples of reforming catalysts that can be used are nickel, nickel oxide, cobalt oxide, chromia, and molybdenum oxide. Optionally, the catalyst is employed with at least one promoter. Examples of promoters include alkali and rare earth promoters. Generally, promoted nickel oxide catalysts are preferred.

The amount of Group 6 or Group 8-10 metals in the catalyst can vary. Preferably, the catalyst includes from about 3 wt % to about 40 wt % of at least one Group 6 or Group 8-10 metal, based on total weight of the catalyst. Preferably, the catalyst includes from about 5 wt % to about 25 wt % of at least one Group 6 or Group 8-10 metal, based on total weight of the catalyst.

The reforming catalyst, optionally, contains one or more metals to suppress carbon deposition during steam reforming. Such metals are selected from the metals of Group 14 and Group 15 of the Periodic Table of the Elements. Preferred Group 14 and Group 15 metals include germanium, tin, lead, arsenic, antimony, and bismuth. Such metals are preferably included in the catalyst in an amount of from about 0.1 wt % to about 30 wt %, based on total weight of nickel in the catalyst.

In a catalyst comprising nickel and/or cobalt there may also be present one or more platinum group metals, which are capable of increasing the activity of the nickel and/or cobalt and of decreasing the tendency to carbon lay-down when reacting steam with hydrocarbons higher than methane. The concentration of such platinum group metal is typically in the range 0.0005 to 0.1% w/w as metal, calculated as the whole catalyst unit. Further, the catalyst, especially in preferred forms, can contain a platinum group metal but no non-noble catalytic component. Such a catalyst is more suitable for the hydrocarbon steam reforming reaction than one containing a platinum group metal on a conventional support because a greater fraction of the active metal is accessible to the reacting gas. A typical content of platinum group metal when used alone is in the range 0.0005 to 0.5% w/w as metal, calculated on the whole catalytic unit.

In one embodiment, the reformer unit includes tubes which are packed with solid catalyst granules. Preferably, the solid catalyst granules comprise nickel or other catalytic agents deposited on a suitable inert carrier material. More preferably, the catalyst is NiO supported on calcium aluminate, alumina, spinel type magnesium aluminum oxide, or calcium aluminate titanate.

In yet another embodiment, both the hydrocarbon feed stream and the steam are preheated prior to entering the reformer. The hydrocarbon feedstock is preheated up to as high a temperature as is consistent with the avoiding of undesired pyrolysis or other heat deterioration. Since steam reforming is endothermic in nature, and since there are practical limits to the amount of heat that can be added by indirect heating in the reforming zones, preheating of the feed is desired to facilitate the attainment and maintenance of a suitable temperature within the reformer itself. Accordingly, it is desirable to preheat both the hydrocarbon feed and the steam to a temperature of at least 200° C.; preferably at least 400° C. The reforming reaction is generally carried out at a reformer temperature of from about 500° C. to about 1,200° C., preferably from about 800° C. to about 1,100° C., and more preferably from about 900° C. to about 1,050° C.

Gas hourly space velocity in the reformer should be sufficient for providing the desired CO to CO₂ balance in the synthesis gas. Preferably, the gas hourly space velocity (based on wet feed) is from about 3,000 per hour to about 10,000 per hour, more preferably from about 4,000 per hour to about 9,000 per hour, and most preferably from about 5,000 per hour to about 8,000 per hour.

Any conventional reformer can be used in the step of catalytic steam reforming. The use of a tubular reformer is preferred. Preferably, the hydrocarbon feed is passed to a tubular reformer together with steam, and the hydrocarbon and steam contact a steam reforming catalyst. In one embodiment, the steam reforming catalyst is disposed in a plurality of furnace tubes that are maintained at an elevated temperature by radiant heat transfer and/or by contact with combustion gases. Fuel, such as a portion of the hydrocarbon feed, is burned in the reformer furnace to externally heat the reformer tubes therein. See, for example, Kirk-Othmer, Encyclopedia of Chemical Technology, 3rd Ed., 1990, Vol. 12, p. 951; and Ullmann's Encyclopedia of Industrial Chemistry, 5th Ed., 1989, Vol. A-12, p. 186, the relevant portions of each being fully incorporated herein by reference.

The ratio of steam to hydrocarbon feed will vary depending on the overall conditions in the reformer. The amount of steam employed is influenced by the requirement of avoiding carbon deposition on the catalyst, and by the acceptable methane content of the effluent at the reforming conditions maintained. On this basis, the mole ratio of steam to hydrocarbon feed in the conventional primary reformer unit is preferably from about 1.5:1 to about 5:1, preferably from about 2:1 to about 4:1.

The hydrogen to carbon oxide ratio of the synthesis gas produced will vary depending on the overall conditions of the reformer. Preferably, the molar ratio of hydrogen to carbon oxide in the synthesis gas will range from about 1:1 to about 5:1. More preferably the molar ratio of hydrogen to carbon oxide will range from about 2:1 to about 3:1. Even more preferably the molar ratio of hydrogen to carbon oxide will range from about 2:1 to about 2.5:1. Most preferably the molar ratio of hydrogen to carbon oxide will range from about 2:1 to about 2.3:1.

Steam reforming is generally carried out at superatmospheric pressure. The specific operating pressure employed is influenced by the pressure requirements of the subsequent process in which the reformed gas mixture is to be employed. Although any superatmospheric pressure can be used in practicing the invention, pressures of from about 175 psig (1,308 kPa abs.) to about 1,100 psig (7,686 kPa abs.) are desirable. Preferably, steam reforming is carried out at a pressure of from about 300 psig (2,170 kPa abs.) to about 800 psig (5,687 kPa abs.), more preferably from about 350 psig (2,515 kPa abs.) to about 700 psig (4,928 kPa abs.).

### C. Partial Oxidation to Make Syngas

The invention further provides for the production of synthesis gas, or CO and H₂, by oxidative conversion (also referred to herein as partial oxidation) of hydrocarbon, particularly natural gas and C₁-C₅ hydrocarbons. According to the process, hydrocarbon is reacted with free-oxygen to form the CO and H₂. The process is carried out with or without a catalyst. The use of a catalyst is preferred, preferably with the catalyst containing at least one non-transition or transition metal oxides. The process is essentially exothermic, and is an incomplete combustion reaction, having the following general formula:

CₙHₘ+(n/2) [symbol] nCO+(m/2)H₂ (4)

Non-catalytic partial oxidation of hydrocarbons to H₂, CO, and CO₂ is desirably used for producing syngas from heavy fuel oils, primarily in locations where natural gas or lighter hydrocarbons, including naphtha, are unavailable or uneconomical compared to the use of fuel oil or crude oil. The non-catalytic partial oxidation process is carried out by injecting preheated hydrocarbon, oxygen and steam through a burner into a closed combustion chamber. Preferably, the individual components are introduced at a burner where they meet in a diffusion flame, producing oxidation products and heat. In the combustion chamber, partial oxidation of the hydrocarbons generally occurs with less than stoichiometric oxygen at very high temperatures and pressures. Preferably, the components are preheated and pressurized to reduce reaction time. The process preferably occurs at a temperature of from about 1,350° C. to about 1,600° C., and at a pressure of from above atmospheric to about 150 atm.

Catalytic partial oxidation comprises passing a gaseous hydrocarbon mixture, and oxygen, preferably in the form of air, over reduced or unreduced catalysts. The reaction is, optionally, accompanied by the addition of water vapor (steam). When steam is added, the reaction is generally referred to as autothermal reduction. Autothermal reduction is both exothermic and endothermic as a result of adding both oxygen and water.

In the partial oxidation process, the catalyst comprises at least one transition element selected from the group consisting of Ni, Co, Pd, Ru, Rh, Ir, Pt, Os, and Fe. Preferably, the catalyst comprises at least one transition element selected from the group consisting of Pd, Pt, and Rh. In another embodiment, preferably the catalyst comprises at least one transition element selected from the group consisting of Ru, Rh, and Ir.

In one embodiment, the partial oxidation catalyst further comprises at least one metal selected from the group consisting of Ti, Zr, Hf, Y, Th, U, Zn, Cd, B, Al, Ti, Si, Sn, Pb, P, Sb, Bi, Mg, Ca, Sr, Ba, Ga, V, and Sc. Also, optionally, included in the partial oxidation catalyst is at least one rare earth element selected from the group consisting of La, Ce, Pr, Nd, Pm, Sm, Eu, Gd, Tb, Dy, Ho, Er, Tm, Yb, and Lu.

In another embodiment the catalyst employed in the process may comprise a wide range of catalytically active components, for example, Pd, Pt, Rh, Ir, Os, Ru, Ni, Cr, Co, Ce, La, and mixtures thereof. Materials not normally considered to be catalytically active may also be employed as catalysts, for example refractory oxides such as cordierite, mullite, mullite aluminum titanate, zirconia spinels, and alumina.

In yet another embodiment, the catalyst is comprised of metals selected from those having atomic number 21 to 29, 40 to 47, and 72 to 79, the metals Sc, Ti V, Cr, Mn, Fe, Co, Ni, Cu, Zr, Nb, Mo, Tc, Ru, Rh, Pd, Ag, Hf, Ta, W, Re, Os Ir, Pt, and Au. The preferred metals are those in Group 8 of the Periodic Table of the Elements, that is Fe, Os, Co, Re, Ir, Pd, Pt, Ni, and Ru.

In another embodiment, the partial oxidation catalyst comprises at least one transition or non-transition metal deposited on a monolith support. The monolith supports are preferably impregnated with a noble metal such as Pt, Pd, or Rh, or other transition metals such as Ni, Co, Cr, and the like. Desirably, these monolith supports are prepared from solid refractory or ceramic materials such as alumina, zirconia, magnesia, ceria, silica, titania, mixtures thereof, and the like. Mixed refractory oxides, that is refractory oxides comprising at least two cations, may also be employed as carrier materials for the catalyst.

In one embodiment, the catalyst is retained in the form of a fixed arrangement. The fixed arrangement generally comprises a fixed bed of catalyst particles. Alternatively, the fixed arrangement comprises the catalyst in the form of a monolith structure. The fixed arrangement may consist of a single monolith structure or, alternatively, may comprise a number of separate monolith structures combined to form the fixed arrangement. A preferred monolith structure comprises a ceramic foam. Suitable ceramic foams for use in the process are available commercially.

In yet another embodiment, the feed comprises methane, and the feed is injected with oxygen into the partial oxidation reformer at a methane to oxygen (i.e., O₂) ratio of from about 1.2:1 to about 10:1. Preferably the feed and oxygen are injected into the reformer at a methane to oxygen ratio of from about 1.6:1 to about 8:1, more preferably from about 1.8:1 to about 4:1.

Water may or may not be added to the partial oxidation process. When added, the concentration of water injected into the reformer is not generally greater than about 65 mole %, based on total hydrocarbon and water feed content. Preferably, when water is added, it is added at a water to methane ratio of not greater than 3:1, preferably not greater than 2:1.

The catalyst may or may not be reduced before the catalytic reaction. In one embodiment, the catalyst is reduced and reduction is carried out by passing a gaseous mixture comprising hydrogen and inert gas (e.g., N₂, He, or Ar) over the catalyst in a fixed-bed reactor at a catalyst reduction pressure of from about 1 atm to about 5 atm, and a catalyst reduction temperature of from about 300° C. to about 700° C. Hydrogen gas is used as a reduction gas, preferably at a concentration of from about 1 mole % to about 100 mole %, based on total amount of reduction gas. Desirably, the reduction is further carried out at a space velocity of reducing gas mixture of from about 10³ cm³/g hr to about 10⁵ cm³/g hr for a period of from about 0.5 hour to about 20 hours.

In one embodiment, the partial oxidation catalyst is not reduced by hydrogen. When the catalyst is not reduced by hydrogen before the catalytic reaction, the reduction of the catalyst can be effected by passing the hydrocarbon feed and oxygen (or air) over the catalyst at a temperature in the range of from about 500° C. to about 900° C. for a period of from about 0.1 hour to about 10 hours.

In the partial oxidation process, carbon monoxide (CO) and hydrogen (H₂) are formed as major products, and water and carbon dioxide (CO₂) as minor products. The above-mentioned products, unconverted reactants (i.e., methane or natural gas and oxygen) and components of feed other than reactants are typically recovered as one or more gas product streams.

When water is added in the feed, the H₂:CO mole ratio in the product is increased by the shift reaction: CO+H₂O [symbol] H₂+CO₂. This reaction occurs simultaneously with the oxidative conversion of the hydrocarbon in the feed to CO and H₂ or synthesis gas. The hydrocarbon used as feed in the partial oxidation process is preferably in the gaseous phase when contacting the catalyst. The partial oxidation process is particularly suitable for the partial oxidation of methane, natural gas, associated gas or other sources of light hydrocarbons. In this respect, the term “light hydrocarbons” is a reference to hydrocarbons having from 1 to 5 carbon atoms. The process may be advantageously applied in the conversion of gas from naturally occurring reserves of methane which contain substantial amounts of carbon dioxide. In one embodiment, the hydrocarbon feed preferably contains from about 10 mole % to about 90 mole % methane, based on total feed content. More preferably, the hydrocarbon feed contains from about 20 mole % to about 80 mole % methane, based on total feed content. In another embodiment, the feed comprises methane in an amount of at least 50% by volume, more preferably at least 70% by volume, and most preferably at least 80% by volume.

In one embodiment of the invention, the hydrocarbon feedstock is contacted with the catalyst in a mixture with an oxygen-containing gas. Air is suitable for use as the oxygen-containing gas. Substantially pure oxygen as the oxygen-containing gas is preferred on occasions where there is a need to avoid handling large amounts of inert gas such as nitrogen. The feed, optionally, comprises steam.

In another embodiment of the invention, the hydrocarbon feedstock and the oxygen-containing gas are preferably present in the feed in such amounts as to give an oxygen-to-carbon ratio in the range of from about 0.3:1 to about 0.8:1, more preferably, in the range of from about 0.45:1 to about 0.75:1. References herein to the oxygen-to-carbon ratio refer to the ratio of oxygen in the form of oxygen molecules (O₂) to carbon atoms present in the hydrocarbon feedstock. Preferably, the oxygen-to-carbon ratio is in the range of from about 0.45:1 to about 0.65:1, with oxygen-to-carbon ratios in the region of the stoichiometric ratio of 0.5:1, that is ratios in the range of from about 0.45:1 to about 0.65:1, being more preferred. When steam is present in the feed, the steam-to-carbon ratio is not greater than about 3.0:1, more preferably not greater than about 2.0:1. The hydrocarbon feedstock, the oxygen-containing gas and steam, if present, are preferably well mixed prior to being contacted with the catalyst.

The partial oxidation process is operable over a wide range of pressures. For applications on a commercial scale, elevated pressures, that is pressures significantly above atmospheric pressure, are preferred. In one embodiment, the partial oxidation process is operated at pressures of greater than atmospheric up to about 150 bars. Preferably, the partial oxidation process is operated at a pressure in the range of from about 2 bars to about 125 bars, more preferably from about 5 bars to about 100 bars.

The partial oxidation process is also operable over a wide range of temperatures. At commercial scale, the feed is preferably contacted with the catalyst at high temperatures. In one embodiment, the feed mixture is contacted with the catalyst at a temperature in excess of 600° C. Preferably, the feed mixture is contacted with the catalyst at a temperature in the range of from about 600° C. to about 1,700° C., more preferably from about 800° C. to about 1,600° C. The feed mixture is preferably preheated prior to contacting the catalyst.

The feed is provided during the operation of the process at a suitable space velocity to form a substantial amount of CO in the product. In one embodiment, gas space velocities (expressed in normal liters of gas per kilogram of catalyst per hour) are in the range of from about 20,000 Nl/kg/hr to about 100,000,000 Nl/kg/hr, more preferably in the range of from about 50,000 Nl/kg/hr to about 50,000,000 Nl/kg/hr, and most preferably in the range of from about 500,000 Nl/kg/hr to about 30,000,000 Nl/kg/hr.

### D. Combination Syngas Processes

Combination reforming processes can also be incorporated into this invention. Examples of combination reforming processes include autothermal reforming and fixed-bed syngas generation. These processes involve a combination of gas phase partial oxidation and steam reforming chemistry.

The autothermal reforming process preferably comprises two synthesis gas generating processes, a primary oxidation process and a secondary steam reforming process. In one embodiment, a hydrocarbon feed stream is steam reformed in a tubular primary reformer by contacting the hydrocarbon and steam with a reforming catalyst to form a hydrogen and carbon monoxide-containing primary reformed gas, the carbon monoxide content of which is further increased in the secondary reformer. In one embodiment, the secondary reformer includes a cylindrical refractory lined vessel with a gas mixer, preferably in the form of a burner in the inlet portion of the vessel and a bed of nickel catalyst in the lower portion. In a more preferred embodiment, the exit gas from the primary reformer is mixed with air and residual hydrocarbons, and the mixed gas partial oxidized to carbon monoxides.

In another embodiment, incorporating the autothermal reforming process, partial oxidation is carried out as the primary oxidizing process. Preferably, hydrocarbon feed, oxygen, and, optionally, steam, are heated and mixed at an outlet of a single large coaxial burner or injector which discharges into a gas phase partial oxidation zone. Oxygen is preferably supplied in an amount which is less than the amount required for complete combustion.

Upon reaction in the partial oxidation combustion zone, the gases flow from the primary reforming process into the secondary reforming process. In one embodiment, the gases are passed over a bed of steam reforming catalyst particles or a monolithic body, to complete steam reforming. Desirably, the entire hydrocarbon conversion is completed by a single reactor aided by internal combustion.

In an alternative embodiment of the invention, a fixed-bed syngas generation process is used to form synthesis gas. In the fixed-bed syngas generation process, hydrocarbon feed and oxygen or an oxygen-containing gas are introduced separately into a fluid catalyst bed. Preferably, the catalyst is comprised of nickel and supported primarily on alpha alumina.

The fixed-bed syngas generation process is carried out at conditions of elevated temperatures and pressures that favor the formation of hydrogen and carbon monoxide when, for example, methane is reacted with oxygen and steam. Preferably, temperatures are in excess of about 1,700° F. (927° C.), but not so high as to cause disintegration of the catalyst or the sticking of catalyst particles together. Preferably, temperatures range from about 1,750° F. (954° C.) to about 1,950° F. (1,066° C.), more preferably, from about 1,800° F. (982° C.) to about 1,850° F. (1,010° C.).

Pressure in the fixed-bed syngas generation process may range from atmospheric to about 40 atmospheres. In one embodiment, pressures of from about 20 atmospheres to about 30 atmospheres are preferred, which allows subsequent processes to proceed without intermediate compression of product gases.

In one embodiment of the invention, methane, steam, and oxygen are introduced into a fluid bed by separately injecting the methane and oxygen into the bed. Alternatively, each stream is diluted with steam as it enters the bed. Preferably, methane and steam are mixed at a methane to steam molar ratio of from about 1:1 to about 3:1, and more preferably from about 1.5:1 to about 2.5:1, and the methane and steam mixture is injected into the bed. Preferably, the molar ratio of oxygen to methane is from about 0.2:1 to about 1.0:1, more preferably from about 0.4:1 to about 0.6:1.

In another embodiment of the invention, the fluid bed process is used with a nickel-based catalyst supported on alpha alumina. In another embodiment, silica is included in the support. The support is preferably comprised of at least 95 wt % alpha alumina, more preferably at least about 98% alpha alumina, based on total weight of the support.

### III. Syngas Feed to the Alcohol Synthesis Process

Synthesis gas (syngas) is used as a feedstock to form a mixed alcohol product. In one embodiment of the invention, the synthesis gas feed (including any recycle syngas recovered from the process itself, as well as fresh syngas) has a molar ratio of hydrogen (H₂) to carbon oxides (CO+CO₂) in the range of from about 0.5:1 to about 20:1, preferably in the range of from about 1:1 to about 10:1. In another embodiment, the synthesis gas has a molar ratio of hydrogen (H₂) to carbon monoxide (CO) of at least 2:1. Carbon dioxide is, optionally, present in an amount of not greater than 50% by weight, based on total weight of the synthesis gas, and preferably less than 20% by weight, more preferably less than 10% by weight.

The stoichiometric molar ratio [i.e., a molar ratio of (H₂+CO₂)/(CO+CO₂)] of the syngas should be sufficiently high so as to maintain a high yield of methanol and ethanol, but not so high as to reduce the volume productivity of both the methanol and ethanol. In one embodiment, the synthesis gas fed to the alcohol synthesis process has a stoichiometric molar ratio of from about 1.0:1 to about 2.7:1, more preferably from about 1.5:1 to about 2.5:1, more preferably a stoichiometric molar ratio of from about 1.7:1 to about 2.5:1.

### IV. Converting Syngas to Mixed Alcohol

There are at least two different catalysts used in converting the syngas to the alcohol product stream in this invention. The first, and preferably two of the catalysts, include an oxide of at least one element selected from the group consisting of copper, silver, zinc, boron, magnesium, aluminum, vanadium, chromium, manganese, gallium, palladium, osmium, and zirconium. More preferably, the first catalyst is a copper-based catalyst. Most preferably at least two of the catalysts, are copper-based catalysts. At least one of the catalyst components is an oxide-containing catalyst that has been modified to contain a Fischer-Tropsch metal (i.e., cobalt, iron, and nickel).

The catalysts can be used in one or more than one reactor. Preferably, the catalysts are used in a single reactor.

The catalysts can be mixed together or staged in separate beds. In one embodiment, at least two different alcohol forming catalysts are used, and at least one of the catalysts is an oxide-containing catalyst that has been modified with a Fischer-Tropsch metal. Preferably, the oxide-containing catalyst that has been modified with a Fischer-Tropsch metal includes an oxide of at least one element selected from the group consisting of copper, silver, zinc, boron, magnesium, aluminum, vanadium, chromium, manganese, gallium, palladium, osmium, and zirconium. More preferably, the oxide-containing catalyst that has been modified with a Fischer-Tropsch metal is a Fischer-Tropsch metal modified copper-based oxide catalyst that includes an oxide of at least one element selected from the group consisting of silver, zinc, boron, magnesium, aluminum, vanadium, chromium, manganese, gallium, palladium, osmium, and zirconium. Still more preferably, the oxide-containing catalyst that has been modified with a Fischer-Tropsch metal is a Fischer-Tropsch metal modified copper-based oxide catalyst that includes an oxide of at least one element selected from the group consisting of zinc, magnesium, aluminum, chromium, and zirconium. Most preferably, the oxide-containing catalyst that has been modified with a Fischer-Tropsch metal is a Fischer-Tropsch metal modified copper-based oxide catalyst that includes zinc oxide. In a particular embodiment, the Fischer-Tropsch metal is cobalt.

In one embodiment, the oxide-containing catalyst that has been modified with a Fischer-Tropsch metal is downstream of at least one other alcohol forming catalyst. Preferably, another of the alcohol forming catalysts includes an oxide of at least one element selected from the group consisting of copper, silver, zinc, boron, magnesium, aluminum, vanadium, chromium, manganese, gallium, palladium, osmium, and zirconium. More preferably, another of the alcohol forming catalysts is a copper-based catalyst that includes an oxide of at least one element selected from the group consisting of silver, zinc, boron, magnesium, aluminum, vanadium, chromium, manganese, gallium, palladium, osmium, and zirconium. Still more preferably, another of the alcohol forming catalysts is a copper-based catalyst that includes an oxide of at least one element selected from the group consisting of zinc, magnesium, aluminum, chromium, and zirconium. Most preferably, another of the alcohol forming catalysts is a copper-based catalyst that includes zinc oxide.

In yet another embodiment, two catalysts are used and the first catalyst is located upstream of the second catalyst. In another embodiment, the first catalyst is located upstream of the second catalyst in a single reactor. In still another embodiment, the first alcohol forming catalyst that contacts the syngas is a copper-based catalyst. Preferably, the first alcohol forming catalyst is a copper-based catalyst that includes an oxide of at least one element selected from the group consisting of silver, zinc, boron, magnesium, aluminum, vanadium, chromium, manganese, gallium, palladium, osmium, and zirconium. More preferably, the first alcohol forming catalyst is a copper-based catalyst that includes an oxide of at least one element selected from the group consisting of zinc, magnesium, aluminum, chromium, and zirconium. Most preferably, the first alcohol forming catalyst is a copper-based catalyst that includes zinc oxide.

In a particular embodiment of the invention, the second alcohol forming catalyst that is used is a Fischer-Tropsch metal modified copper-based oxide catalyst. Preferably, the modified copper-based oxide catalyst includes an oxide of at least one element selected from the group consisting of silver, zinc, boron, magnesium, aluminum, vanadium, chromium, manganese, gallium, palladium, osmium, and zirconium. More preferably, the modified copper-based oxide catalyst includes an oxide of at least one element selected from the group consisting of zinc, magnesium, aluminum, chromium, and zirconium. Most preferably, the modified copper-based oxide catalyst includes zinc oxide.

According to this invention Fischer-Tropsch metals include at least one metal selected from the group consisting of cobalt, iron, and nickel. Cobalt represents a preferred Fischer-Tropsch metal in this invention.

In one embodiment of the invention, at least one of the catalysts, preferably a first and second catalyst, is a copper-based oxide catalyst. Preferably, the copper oxide-based catalysts used in this invention comprise from about 10 wt % to about 70 wt % copper oxide, based on total weight of the catalyst. More preferably, the copper oxide-based catalysts used in this invention comprise from about 15 wt % to about 68 wt % copper oxide, and most preferably from about 20 wt % to about 65 wt % copper oxide, based on total weight of the catalyst.

In another embodiment of the invention, at least one of the catalysts, preferably a first and second catalyst comprise zinc oxide. Preferably, the zinc oxide-containing catalysts comprise from about 3 wt % to about 40 wt % zinc oxide, based on total weight of the catalyst. More preferably, the zinc oxide-containing catalysts comprise from about 4 wt % to about 35 wt % zinc oxide, and most preferably, from about 5 wt % to about 30 wt % zinc oxide.

In embodiments in which copper oxide and zinc oxide are both present in at least one of the catalysts, the ratio of copper oxide to zinc oxide comprises copper oxide and zinc oxide in a Cu:Zn atomic ratio of from about 0.5:1 to about 20:1, preferably from about 0.7:1 to about 15:1, more preferably from about 0.8:1 to about 5:1.

The conversion of syngas to alcohol product can be accomplished over a wide range of temperatures. Lower temperature ranges are preferred. In one embodiment, the syngas is contacted with the catalysts at a temperature in the range of from about 150° C. to about 450° C., preferably in a range of from about 175° C. to about 350° C., more preferably in a range of from about 200° C. to about 300° C.

The syngas can also be converted to alcohol product over a wide range of pressures. In one embodiment, the syngas is contacted with the catalyst at a pressure in the range of from about 15 atmospheres to about 125 atmospheres, preferably in a range of from about 20 atmospheres to about 100 atmospheres, more preferably in a range of from about 25 atmospheres to about 75 atmospheres.

Gas hourly space velocities in converting the syngas to alcohol product can vary depending upon the type of reactor that is used. In one embodiment, gas hourly space velocity of flow of gas through the catalyst bed is in the range of from about 50 hr⁻¹ to about 50,000 hr⁻¹. Preferably, gas hourly space velocity of flow of gas through the catalyst bed is in the range of from about 250 hr⁻¹ to about 25,000 hr⁻¹, more preferably from about 500 hr⁻¹ to about 20,000 hr⁻¹.

### V. Recovery and Further Processing of Methanol Product

The methanol product from the fluidized-bed reactor is generally sent to a separation unit or vessel to remove light product having a higher boiling point than the methanol. This separation preferably yields a liquid product rich in methanol, although the separated methanol product can include other components such as water. The separated methanol product can be used “as is,” or it can be further processed if desired. Processing can be accomplished using any conventional means. Examples of such means include distillation, selective condensation, and selective adsorption. Process conditions, e.g., temperatures and pressures, can vary according to the particular methanol composition desired. It is particularly desirable to minimize the amount of water and light boiling point components in the methanol composition, but without substantially reducing the amount of methanol present.

In one embodiment, the separated and recovered methanol product is sent to a let down vessel so as to reduce the pressure to about atmospheric or slightly higher. This let down in pressure allows undesirable light boiling point components to be removed from the methanol composition as a vapor. The vapor is desirably of sufficient quality to use as fuel.

In another embodiment, the separated recovered methanol product is sent from the methanol synthesizing unit or vessel to a distillation system. The distillation system contains one or more distillation columns which are used to further separate the desired methanol composition from water and hydrocarbon by-product streams. Desirably, the methanol composition that is separated from the crude methanol comprises a majority of the methanol contained in the methanol product prior to separation.

In one embodiment, the distillation system includes a step of treating the recovered methanol product steam being distilled so as to remove or neutralize acids in the stream. Preferably, a base is added in the system that is effective in neutralizing organic acids that are found in the methanol stream. Conventional base compounds can be used. Examples of base compounds include alkali metal hydroxide or carbonate compounds, and amine or ammonium hydroxide compounds. In one particular embodiment, about 20 ppm to about 120 ppm w/w of a base composition, calculated as stoichiometrically equivalent NaOH, is added, preferably about 25 ppm to about 100 ppm w/w of a base composition, calculated as stoichiometrically equivalent NaOH, is added.

Examples of distillation systems include the use of single and two column distillation columns. Preferably, the single columns operate to remove volatiles in the overhead, methanol product at a high level, fusel oil as vapor above the feed and/or as liquid below the feed, and water as a bottoms stream.

In one embodiment of a two column system, the first column is a “topping column” from which volatiles are taken overhead and methanol liquid as bottoms. The second is a “rectifying column” from which methanol product is taken as an overhead stream or at a high level, and water is removed as a bottoms stream. In this embodiment, the rectifying column includes at least one off-take for fusel oil as vapor above the feed and/or as liquid below the feed.

In another embodiment of a two column system, the first column is a water-extractive column in which there is a water feed introduced at a level above the crude methanol feed level. It is desirable to feed sufficient water to produce a bottoms liquid containing over 40% w/w water, preferably 40% to 60% w/w water, and more preferably 80% to 95% w/w water. This column, optionally, includes one or more direct fusel oil side off-takes.

In yet another embodiment, the distillation system is one in which an aqueous, semi-crude methanol is taken as liquid above the feed in a single or rectifying column. The semi-crude methanol is passed to a rectifying column, from which methanol product is taken overhead or at a high level. Preferably, water or aqueous methanol is taken as a bottoms stream.

Alternatively, undesirable by-products are removed from the separated methanol stream from the methanol synthesis reactor by adsorption. In such a system, other components such as fusel oil can be recovered by regenerating the adsorbent.

### VI. Converting the Alcohol Composition to Olefins

### A. General Process Description

In one embodiment of the invention, the alcohol composition is converted to olefins by contacting the alcohol composition with an olefin forming catalyst to form the olefin product. The olefin product is recovered, and water, which forms during the conversion of the oxygenates in the methanol to olefins, is removed. After removing the water, the olefins are separated into individual olefin streams, and each individual olefin stream is available for further processing.

### B. Description of Olefin Forming Catalyst

Any catalyst capable of converting oxygenate to olefin can be used in this invention. Molecular sieve catalysts are preferred. Examples of such catalysts include zeolite as well as non-zeolite molecular sieves, and are of the large, medium, or small pore type. Non-limiting examples of these molecular sieves are the small pore molecular sieves, AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves, AFO, AEL, EUO, HEU, FER, MEL, MFI, MTW, MTT, TON, and substituted forms thereof; and the large pore molecular sieves, EMT, FAU, and substituted forms thereof. Other molecular sieves include ANA, BEA, CFI, CLO, DON, GIS, LTL, MER, MOR, MWW, and SOD. Non-limiting examples of the preferred molecular sieves, particularly for converting an oxygenate containing feedstock into olefin(s), include AEL, AFY, BEA, CHA, EDI, FAU, FER, GIS, LTA, LTL, MER, MFI, MOR, MTT, MWW, TAM, and TON. In one preferred embodiment, the molecular sieve of the invention has an AEI topology or a CHA topology, or a combination thereof, most preferably a CHA topology.

In one embodiment, aluminophosphate (ALPO) molecular sieves, silicoaluminophosphate (SAPO) molecular sieves or a combination thereof is used. Preferred molecular sieves are SAPO molecular sieves, and metal substituted SAPO molecular sieves. In an embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn, and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

Non-limiting examples of SAPO and ALPO molecular sieves used in the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18, and ALPO-34, even more preferably one or a combination of SAPO-18, SAPO-34, ALPO-34, and ALPO-18, and metal containing molecular sieves thereof, and most preferably one or a combination of SAPO-34 and ALPO-18, and metal containing molecular sieves thereof.

In an embodiment, the molecular sieve is an intergrowth material having two or more distinct phases of crystalline structures within one molecular sieve composition. In particular, intergrowth molecular sieves are described in the U.S. Pat. No. 6,813,372 and PCT WO 98/15496 published Apr. 16, 1998, both of which are fully incorporated herein by reference. In another embodiment, the molecular sieve comprises at least one intergrown phase of AEI and CHA framework types. For example, SAPO-18, ALPO-18, and RUW-18 have an AEI framework type, and SAPO-34 has a CHA framework type.

The molecular sieves are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by conventional techniques such as spray drying, pelletizing, extrusion, and the like.

### C. General Conditions for Converting Alcohol to Olefins

According to the reaction process of this invention, the mixed alcohol stream is contacted with olefin forming catalyst to form an olefin product, particularly ethylene and propylene. The process for converting the oxygenate feedstock is, preferably, a continuous fluidized-bed process, and most preferably a continuous high velocity fluidized-bed process.

The reaction processes can take place in a variety of catalytic reactors such as hybrid reactors that have dense bed or fixed-bed reaction zones and/or fast fluidized-bed reaction zones coupled together, circulating fluidized-bed reactors, riser reactors, and the like. Suitable conventional reactor types are described in, for example, U.S. Pat. Nos. 4,076,796 and 6,287,522 (dual riser), and Fluidization Engineering, D. Kunii and O. Levenspiel, Robert E. Krieger Publishing Company, New York, N.Y. 1977.

One preferred reactor type is a riser reactor. These types of reactors are generally described in Riser Reactor, Fluidization and Fluid-Particle Systems, pp. 48 to 59, F. A. Zenz and D. F. Othmo, Reinhold Publishing Corporation, New York, 1960, and U.S. Pat. No. 6,166,282 (fast-fluidized-bed reactor).

In one embodiment of the invention, a fluidized-bed process or high velocity fluidized-bed process includes a reactor system, catalyst separation system, and a regeneration system. The reactor system preferably is a fluid bed reactor system. In one embodiment, the fluid bed reactor system has a first reaction zone within one or more riser reactors, and a second reaction zone within at least one catalyst separation vessel, preferably comprising one or more cyclones. In one embodiment, one or more riser reactors and catalyst separation vessel is contained within a single reactor vessel.

The average reaction temperature employed in the conversion process, specifically within the reactor, is of from about 250° C. to about 800° C. Preferably the average reaction temperature within the reactor is from about 250° C. to about 750° C.; more preferably, from about 300° C. to about 650° C.; yet more preferably from about 350° C. to about 600° C.; and most preferably from about 400° C. to about 500° C.

The pressure employed in the conversion process, specifically within the reactor, is not critical. The reaction pressure is based on the partial pressure of the feedstock exclusive of any diluent therein. Typically, the reaction pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), defined as the total weight of the feedstock excluding any diluents to the reaction zone per hour per weight of molecular sieve in the molecular sieve catalyst composition in the reaction zone, is maintained at a level sufficient to keep the catalyst composition in a fluidized state within a reactor. Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr-1.

The superficial gas velocity (SGV) of the feedstock, including diluent and reaction products within the reactor, is preferably sufficient to fluidize the molecular sieve catalyst composition within a reaction zone of the reactor. The SGV in the process, particularly within the reactor system, more particularly within a riser reactor, is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec.

### VII. Olefin Product Recovery and Use

In one embodiment, olefin product and other gases are withdrawn from the reactor and are passed through a recovery system. Any conventional recovery system, technique and/or sequence useful in separating olefin(s) and purifying olefin(s) from other gaseous components can be used in this invention. Examples of recovery systems include one or more or a combination of various separation, fractionation and/or distillation towers, columns, and splitters, and other associated equipment; for example, various condensers, heat exchangers, refrigeration systems or chill trains, compressors, knock-out drums or pots, pumps, and the like.

Non-limiting examples of distillation towers, columns, splitters or trains used alone or in combination include one or more of a demethanizer, preferably a high temperature demethanizer, a deethanizer, a depropanizer, preferably a wet depropanizer, a wash tower often referred to as a caustic wash tower and/or quench tower, absorbers, adsorbers, membranes, ethylene (C₂) splitter, propylene (C₃) splitter, butene (C₄) splitter, and the like.

Generally accompanying most recovery systems is the production, generation or accumulation of additional products, by-products and/or contaminants along with the preferred prime products. The preferred prime products, the light olefins, such as ethylene and propylene, are typically purified for use in derivative manufacturing processes such as polymerization processes.

The ethylene and propylene streams produced and recovered according to this invention can be polymerized to form plastic compositions, e.g., polyolefins, particularly polyethylene and polypropylene. Any process capable of forming polyethylene or polypropylene can be used. Catalytic processes are preferred. Particularly preferred are metallocene, Ziegler/Natta, aluminum oxide and acid catalytic systems. In general, these methods involve contacting the ethylene or propylene product with a polyolefin-forming catalyst at a pressure and temperature effective to form the polyolefin product.

In one embodiment of this invention, the ethylene or propylene product is contacted with a metallocene catalyst to form a polyolefin. Desirably, the polyolefin forming process is carried out at a temperature ranging between about 50° C. and about 320° C. The reaction can be carried out at low, medium, or high pressure, being anywhere within the range of about 1 bar to about 3200 bar. For processes carried out in solution, an inert diluent can be used. In this type of operation, it is desirable that the pressure be at a range of from about 10 bar to about 150 bar, and preferably at a temperature range of from about 120° C. to about 250° C. For gas phase processes, it is preferred that the temperature generally be within a range of about 60° C. to 120° C., and that the operating pressure be from about 5 bar to about 50 bar.

In addition to polyolefins, numerous other olefin derivatives may be formed from the ethylene, propylene and C₄+ olefins, particularly butylene, separated according to this invention. The olefins separated according to this invention can also be used in the manufacture of such compounds as aldehydes, acids such as C₂-C₁₃ mono carboxylic acids, alcohols such as C₂-C₁₂ mono alcohols, esters made from the C₂-C₁₂ mono carboxylic acids and the C₂-C₁₂ mono alcohols, linear alpha olefins, vinyl acetate, ethylene dicholoride and vinyl chloride, ethylbenzene, ethylene oxide, cumene, acrolein, allyl chloride, propylene oxide, acrylic acid, ethylene-propylene rubbers, and acrylonitrile, and trimers and dimers of ethylene and propylene. The C₄+ olefins, butylene, in particular, are particularly suited for the manufacture of aldehydes, acids, alcohols, esters made from C₅-C₁₃ mono carboxylic acids and C₅-C₁₃ mono alcohols and linear alpha olefins.

### VIII. Examples

### Example 1

This example describes the preparation of an alcohol conversion catalyst (A) having the following composition:

60% CuO/30% ZnO/10% Al₂O₃ (nominal)

The following solutions were prepared:

2 M Cu: 36.4 g Cu(NO₃)₂*3H₂O were dissolved in 75 cc d.i. H₂O

2 M Zn: 22.0 g Zn(NO₃)₂*6H₂O were dissolved in 37 cc d.i. H₂O

2 M Al: 14.8 g Al(NO₃)₃*9H₂O were dissolved in 20 cc d.i. H₂O

2 M Na₂CO₃: 93.0 g Na₂CO₃*H₂O were dissolved in 375 cc d.i. H₂O

A 2 liter flask was filled with 500 ml d.i. H₂O. The water was heated to 70° C. and stirred. The Cu, Zn, and Al solutions were mixed to form solution 1. The Na₂CO₃ solution was labeled as solution 2. While stirring the water and maintaining its temperature at 70° C., solutions 1 and 2 were added simultaneously to the water with pH controlled during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (ca. 1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped 5) cool down to room temperature

### Example 2

This example describes the preparation of an alcohol conversion catalyst (B) having the following composition:

40% CuO/20% CoO/30% ZnO/10% Al₂O₃ (nominal)

The following solutions were prepared:

2 M Cu: 24.3 g Cu(NO₃)₂*3H₂O were dissolved in 50 cc d.i. H₂O

2 M Co: 15.5 g Co(NO₃)₂*6H₂O were dissolved in 27 cc d.i. H₂O

2 M Zn: 21.9 g Zn(NO₃)₂*6H₂O were dissolved in 37 cc d.i. H₂O

2 M Al: 14.7 g Al(NO₃)₃*9H₂O were dissolved in 20 cc d.i. H₂O

2 M Na₂CO₃: 93.0 g Na₂CO₃*H₂O were dissolved in 375 cc d.i. H₂O

A 2 liter flask was filled with 500 ml d.i. H₂O. The water was heated to 70° C. and stirred. The Cu, Co, Zn, and Al solutions were mixed to form a solution 1. The Na₂CO₃ solution was labeled as solution 2. While stirring the water and maintaining its temperature at 70° C., solutions 1 and 2 were added simultaneously to the water with pH controlled during addition at 7.0. The reaction was considered complete when all of solution 1 had been added. The mixture was kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 3

This example describes the preparation of an alcohol conversion catalyst (B2) having the following composition:

45% CuO/15% CoO/30% ZnO/10% Al₂O₃ (nominal)

The following solutions were prepared:

2 M Cu: 27.3 g Cu(NO₃)₂*3H₂O were dissolved in 57 cc d.i. H₂O

2 M Co: 11.7 g Co(NO₃)₂*6H₂O were dissolved in 20 cc d.i. H₂O

2 M Zn: 21.9 g Zn(NO₃)₂*6H₂O were dissolved in 37 cc d.i. H₂O

2 M AI: 14.7 g AI(NO₃)₃*9H₂O were dissolved in 20 cc d.i. H₂O

2 M Na₂CO₃: 93.0 g Na₂CO₃*H₂O were dissolved in 375 cc d.i. H₂O

A 2 liter flask was filled with 500 ml d.i. H₂O. The water was heated to 70° C. and stirred. The Cu, Co, Zn, and Al solutions were mixed to form a solution 1. The Na₂CO₃ solution was labeled as solution 2. While stirring the water and maintaining its temperature at 70° C., solutions 1 and 2 were added simultaneously to the water with pH controlled during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 4

2 ml of catalyst A were loaded in a reactor and tested for syngas conversion under the following conditions: T=300° C., P=750 psi, GHSV=5000, feed composition: 60% H₂, 25% CO, 5% CO₂, 10% N₂. Average conversions and selectivities over a 20-hour period were as follows:

COx conversion=20.7%

Methanol selectivity=64.7%

Ethanol selectivity=2.8%

n-Propanol selectivity=1.5%

1-Butanol selectivity=0.4%

i-Butanol selectivity=2.9%

Methane selectivity=2.0%

Ethane selectivity=3.2%

Methyl formate selectivity=0.4%

Acetaldehyde selectivity=0.6%

DME selectivity=5.6%

Other C₂s, C₃s, C₄s, and C₅+=balance

As indicated, the ethanol selectivity is 2.8% and the isobutanol selectivity (branched alcohol) is 2.9%. The selectivity to methane is fairly low at 2.0%. The results are summarized in Table 1.

### Example 5

2 ml of catalyst B1 were loaded in a reactor and tested for syngas conversion under the following conditions: T=275° C., P=750 psi, GHSV=5000, feed composition: 60% H₂, 25% CO, 5% CO₂, 10% N₂. Average conversions and selectivities over a 20-hour period were as follows:

COx conversion=11.7%

Methanol selectivity=9.9%

Ethanol selectivity=6.0%

n-Propanol selectivity=2.2%

1-Butanol selectivity=1.1%

i-Butanol selectivity=0.1%

Methane selectivity=30.0%

Ethane selectivity=8.6%

Methyl formate selectivity=0.2%

Acetaldehyde selectivity=2.3%

DME selectivity=5.7%

Other C₂s, C₃s, C₄s, and C₅+=balance

Relative to Example 4, the ethanol selectivity showed an increase (6.0%) and the isobutanol selectivity (branched alcohol) was relatively low (0.1%), but the selectivity to paraffins was relatively high (30.0% to methane and 8.6% to ethane). The results are summarized in Table 1.

### Example 6

2 ml of catalyst B2 were loaded in a reactor and tested for syngas conversion under the following conditions: T=275° C., P=750 psi, GHSV=5000, feed composition: 60% H₂, 25% CO, 5% CO₂, 10% N₂. Average conversions and selectivities over a 20-hour period were as follows:

COx conversion=9.0%

Methanol selectivity=11.7%

Ethanol selectivity=6.1%

n-Propanol selectivity=1.9%

1-Butanol selectivity=0.9%

i-Butanol selectivity=0.1%

Methane selectivity=21.1%

Ethane selectivity=6.4%

Methyl formate selectivity=0.1%

Acetaldehyde selectivity=1.2%

DME selectivity=3.1%

Other C₂s, C₃s, C₄s, and C₅+=balance

Again the ethanol selectivity is higher (6.1%) than in Example 4, using catalyst A, and the isobutanol selectivity (branched alcohol) is comparatively lower (0.1%), but the selectivity to paraffins is relatively high (21.1% to methane and 6.4% to ethane). The results are summarized in Table 1.

### Example 7 (Invention)

1 ml of catalyst A was loaded in a reactor on top of 1 ml of catalyst B1 and the two materials were separated by a thin bed of glass wool. This embodiment is shown in the FIGURE. According to the FIGURE, feed containing 60% H₂, 25% CO, 5% CO₂, 10% N₂ was flowed into the reactor (10) to contact a layer of catalyst A (12), with the effluent from the catalyst A layer (12) being flowed across a glass wool layer (14), which is located between the catalyst A layer and the catalyst B1 layer (16). The following reactor (10) conditions were maintained: T=275° C., P=750 psi, GHSV=5000. Average conversions and selectivities over a 22-hour period were as follows:

COx conversion=8.3%

Methanol selectivity=57.4%

Ethanol selectivity=4.9%

n-Propanol selectivity=2.4%

1-Butanol selectivity=0.9%

i-Butanol selectivity=1.3%

Methane selectivity=1.2%

Ethane selectivity=0.8%

Methyl formate selectivity=0.6%

Acetaldehyde selectivity=0.3%

DME selectivity=2.3%

Other C₂s, C₃s, C₄s, and C₅+=balance

As indicated, the ethanol selectivity is higher than in Example 4 using catalyst A, while isobutanol selectivity (branched alcohol) remains relatively low (1.3%), but selectivity to paraffins is very low (1.2% to methane and 0.8% to ethane) using catalyst A and B1 in combination. The results are summarized in Table 1.

### Example 8 (Invention)

1 ml of catalyst A was loaded in a reactor on top of 1 ml of catalyst B2 and the two materials were separated by a thin bed of glass wool. This embodiment is shown in the FIGURE. According to the FIGURE, feed containing 60% H₂, 25% CO, 5% CO₂, 10% N₂ was flowed into the reactor (10) to contact a layer of catalyst A (12), with the effluent from the catalyst A layer (12) being flowed across a glass wool layer (14), which is located between the catalyst A layer and the catalyst B2 layer (16). The following reactor (10) conditions were maintained: T=275° C., P=750 psi, GHSV=5000. Average conversions and selectivities over a 22-hour period were as follows:

COx conversion=8.5%

Methanol selectivity=58.4%

Ethanol selectivity=4.6%

n-Propanol selectivity=2.3%

1-Butanol selectivity=0.9%

i-Butanol selectivity=1.2%

Methane selectivity=1.0%

Ethane selectivity=0.7%

Methyl formate selectivity=0.7%

Acetaldehyde selectivity=0.3%

DME selectivity=2.6%

Other C₂s, C₃s, C₄s, and C₅+=balance

The results indicate that ethanol selectivity is higher than in Example 4 using catalyst A, while isobutanol selectivity (branched alcohol) remained low (1.2%), and selectivity to paraffins was very low (1.0% to methane and 0.7% to ethane). The results are summarized in Table 1.

TABLE 1

| | CATALYST | | | | |
|---|---|---|---|---|---|
| | A | B1 | B2 | A + B1 | A + B2 |
| | (Ex. 4) | (Ex. 5) | (Ex. 6) | (Ex. 7) | (Ex. 8) |
| Loading (ml) | 2 | 2 | 2 | 1 + 1 | 1 + 1 |
| Temperature (° C.) | 300 | 275 | 275 | 275 | 275 |
| Pressure (psi) | 750 | 750 | 750 | 750 | 750 |
| GHSV | 5000 | 5000 | 5000 | 5000 | 5000 |
| CO/H₂/ | 25/60/ | 25/60/ | 25/60/ | 25/60/ | 25/60/ |
| CO₂/N₂ (%) | 5/10 | 5/10 | 5/10 | 5/10 | 5/10 |
| COx conversion (%) | 20.7 | 11.7 | 9 | 8.3 | 8.5 |
| MeOH selectivity (%) | 64.7 | 9.9 | 11.7 | 57.4 | 58.4 |
| EtOH selectivity (%) | 2.8 | 6 | 6.1 | 4.9 | 4.6 |
| n-PrOH selectivity (%) | 1.5 | 2.2 | 1.9 | 2.4 | 2.3 |
| 1-BuOH selectivity (%) | 0.4 | 1.1 | 0.9 | 0.9 | 0.9 |
| i-BuOH selectivity (%) | 2.9 | 0.1 | 0.1 | 1.3 | 1.2 |
| CH₄ selectivity (%) | 2 | 30 | 21.1 | 1.2 | 1 |
| C₂H₆ selectivity (%) | 3.2 | 8.6 | 6.4 | 0.8 | 0.7 |
| MeF selectivity (%) | 0.4 | 0.2 | 0.1 | 0.6 | 0.7 |
| Acetal. selectivity (%) | 0.6 | 2.3 | 1.2 | 0.3 | 0.3 |
| DME selectivity (%) | 5.6 | 5.7 | 3.1 | 2.3 | 2.6 |

The principles and modes of operation of this invention have been described above with reference to various exemplary and preferred embodiments. As understood by those of skill in the art, the overall invention, as defined by the claims, encompasses other preferred embodiments not specifically enumerated herein.

What is claimed is:

1. A process for producing olefin product from syngas, comprising:
contacting at least two different alcohol forming catalysts with a syngas feed to form an alcohol product, wherein at least one of the catalysts is an oxide-containing catalyst that has been modified with a Fischer-Tropsch metal; and
contacting the alcohol product with an olefin forming catalyst to form the olefin product.

2. The process of claim 1, wherein the oxide-containing catalyst that has been modified with a Fischer-Tropsch metal includes an oxide of at least one element selected from the group consisting of copper, silver, zinc, boron, magnesium, aluminum, vanadium, chromium, manganese, gallium, palladium, osmium, and zirconium.

3. The process of claim 2, wherein the oxide-containing catalyst that has been modified with a Fischer-Tropsch metal is a Fischer-Tropsch metal modified copper-based oxide catalyst that includes an oxide of at least one element selected from the group consisting of silver, zinc, boron, magnesium, aluminum, vanadium, chromium, manganese, gallium, palladium, osmium, and zirconium.

4. The process of claim 3, wherein the oxide-containing catalyst that has been modified with a Fischer-Tropsch metal is a Fischer-Tropsch metal modified copper-based oxide catalyst that includes an oxide of at least one element selected from the group consisting of zinc, magnesium, aluminum, chromium, and zirconium.

5. The process of claim 4, wherein the oxide-containing catalyst that has been modified with a Fischer-Tropsch metal is a Fischer-Tropsch metal modified copper-based oxide catalyst that includes zinc oxide.

6. The process of claim 1, wherein the Fischer-Tropsch metal is cobalt.

7. The process of claim 1, wherein the oxide-containing catalyst that has been modified with a Fischer-Tropsch metal is downstream of at least one other alcohol forming catalyst.

8. The process of claim 7, wherein another of the alcohol forming catalysts includes an oxide of at least one element selected from the group consisting of copper, silver, zinc, boron, magnesium, aluminum, vanadium, chromium, manganese, gallium, palladium, osmium, and zirconium.

9. The process of claim 8, wherein another of the alcohol forming catalysts is a copper-based catalyst that includes an oxide of at least one element selected from the group consisting of silver, zinc, boron, magnesium, aluminum, vanadium, chromium, manganese, gallium, palladium, osmium, and zirconium.

10. The process of claim 9, wherein another of the alcohol forming catalysts is a copper-based catalyst that includes an oxide of at least one element selected from the group consisting of zinc, magnesium, aluminum, chromium, and zirconium.

11. The process of claim 10, wherein another of the alcohol forming catalysts is a copper-based catalyst that includes zinc oxide.

12. The process of claim 11, wherein at least one olefin component in the olefin product is contacted with a polyolefin-forming catalyst to form a polyolefin product.

13. A process for producing olefin product from syngas, comprising:
contacting first and second alcohol forming catalysts with a syngas feed to form an alcohol product, wherein the first catalyst includes an oxide of at least one element selected from the group consisting of copper, silver, zinc, boron, magnesium, aluminum, vanadium, chromium, manganese, gallium, palladium, osmium, and zirconium, and the second alcohol forming catalyst is a Fischer-Tropsch metal modified oxide catalyst, wherein the oxide is an oxide of at least one element selected from the group consisting of copper, silver, zinc, boron, magnesium, aluminum, vanadium, chromium, manganese, gallium, palladium, osmium, and zirconium; and
contacting the alcohol product with an olefin forming catalyst to form the olefin product.

14. The process of claim 13, wherein the first alcohol forming catalyst is a copper-based catalyst that includes an oxide of at least one element selected from the group consisting of silver, zinc, boron, magnesium, aluminum, vanadium, chromium, manganese, gallium, palladium, osmium, and zirconium.

15. The process of claim 14, wherein the first alcohol forming catalyst is a copper-based catalyst that includes an oxide of at least one element selected from the group consisting of zinc, magnesium, aluminum, chromium, and zirconium.

16. The process of claim 15, wherein the first alcohol forming catalyst is a copper-based catalyst that includes zinc oxide.

17. The process of claim 13, wherein the second alcohol forming catalyst is a Fischer-Tropsch metal modified copper-based oxide catalyst that includes an oxide of at least one element selected from the group consisting of silver, zinc, boron, magnesium, aluminum, vanadium, chromium, manganese, gallium, palladium, osmium, and zirconium.

18. The process of claim 17, wherein the second alcohol forming catalyst is a Fischer-Tropsch metal modified copper-based oxide catalyst that includes an oxide of at least one element selected from the group consisting of zinc, magnesium, aluminum, chromium, and zirconium.

19. The process of claim 18, wherein the second alcohol forming catalyst is a Fischer-Tropsch metal modified copper-based oxide catalyst that includes zinc oxide.

20. The process of claim 13, wherein the Fischer-Tropsch metal is cobalt.

21. The process of claim 13, wherein the syngas is contacted with the first and second catalysts at a temperature of from 150° C. to 450° C.

22. The process of claim 13, wherein the first and second catalysts are mixed together.

23. The process of claim 13, wherein the first catalyst is located upstream of the second catalyst.

24. The process of claim 13, wherein the first and second catalysts are located in a single reactor.

25. The process of claim 13, wherein at least one olefin component in the olefin product is contacted with a polyolefin-forming catalyst to form a polyolefin product.

26. A process for producing olefin product from syngas, comprising:
contacting syngas with first and second alcohol forming catalysts in a single reactor to form an alcohol product, wherein the firsts catalyst includes an oxide of at least one element selected from the group consisting of copper, silver, zinc, boron, magnesium, aluminum, vanadium, chromium, manganese, gallium, palladium, osmium, and zirconium, and the second alcohol forming catalyst is a Fischer-Tropsch metal modified oxide catalyst, wherein the oxide is an oxide of at least one element selected from the group consisting of copper, silver, zinc, boron, magnesium, aluminum, vanadium, chromium, manganese, gallium, palladium, osmium, and zirconium; and
contacting the alcohol product with an olefin forming catalyst to form the olefin product.

27. The process of claim 26, wherein the first alcohol forming catalyst is a copper-based catalyst that includes an oxide of at least one element selected from the group consisting of silver, zinc, boron, magnesium, aluminum, vanadium, chromium, manganese, gallium, palladium, osmium, and zirconium.

28. The process of claim 27, wherein the first alcohol forming catalyst is a copper-based catalyst that includes an oxide of at least one element selected from the group consisting of zinc, magnesium, aluminum, chromium, and zirconium.

29. The process of claim 28, wherein the first alcohol forming catalyst is a copper-based catalyst that includes zinc oxide.

30. The process of claim 26, wherein the second alcohol forming catalyst is a Fischer-Tropsch metal modified copper-based oxide catalyst that includes an oxide of at least one element selected from the group consisting of silver, zinc, boron, magnesium, aluminum, vanadium, chromium, manganese, gallium, palladium, osmium, and zirconium.

31. The process of claim 30, wherein the second alcohol forming catalyst is a Fischer-Tropsch metal modified copper-based oxide catalyst that includes an oxide of at least one element selected from the group consisting of zinc, magnesium, aluminum, chromium, and zirconium.

32. The process of claim 31, wherein the second alcohol forming catalyst is a Fischer-Tropsch metal modified copper-based oxide catalyst that includes zinc oxide.

33. The process of claim 26, wherein the Fischer-Tropsch metal is cobalt.

34. The process of claim 26, wherein the syngas is contacted with the first and second catalysts at a temperature of from 150° C. to 450° C.

35. The process of claim 26, wherein the first and second catalysts are mixed together in the reactor.

36. The process of claim 26, wherein the first catalyst is located upstream of the second catalyst in the reactor.

37. The process of claim 26, wherein at least one olefin component in the olefin product is contacted with a polyolefin-forming catalyst to form a polyolefin product.
